(19) United States
(12) Patent Application Publication
(10) Pub. No.: US 2020/0297275 A1
(43) Pub. Date: Sep. 24, 2020

(54) Method for the Prevention of Brain Damage after Traumatic Brain Injury by Pharmacological Enhancement of KCNQ Potassium Ion Channels in Neurons

(71) Applicant: Mark S. Shapiro, San Antonio, TX (US)

(72) Inventor: Mark S. Shapiro, San Antonio, TX (US)

(21) Appl. No.: 16/749,960

(22) Filed: Jan. 22, 2020

Related U.S. Application Data
(60) Provisional application No. 62/822,752, filed on Mar. 22, 2019.

(51) Int. Cl. A61B 5/00; A61K 31/222; A61B 5/04

(52) U.S. Cl. CPC A61B 5/4848; A61B 5/04001; A61K 31/222; A61B 5/4064

(57) ABSTRACT

A method for the prevention of brain damage and dysfunction after blunt or blast types of TBI by a single systemic dose application either intravenous (i.v.) or intraperitoneal (i.p.) of a pharmacological “opener” of KCNQ (“M-type”) potassium ion channels in brain.

### CROSS REFERENCE TO RELATED APPLICATIONS

This original non-provisional application claims priority to and the benefit of U.S. provisional application Ser. No. 62/822,752, filed Mar. 22, 2019, and entitled “Strategies of Targeting Electrical Signaling Proteins of Neurons to Prevent Acquired Epilepsies and Brain Dysfunction after Traumatic Brain Injury,” which is incorporated by reference herein.

### STATEMENT REGARDING FEDERALLY SPONSORED RESEARCH OR DEVELOPMENT

This invention was made with Government support under Grant No. W81XWH-15-1-0284 awarded by the U.S. Department of Defense. The Government has certain rights in this invention.

### BACKGROUND OF THE INVENTION

### 1. Field of the Invention

The present invention relates to traumatic brain injury (TBI). More specifically, the present invention relates to a method for the prevention of brain damage and dysfunction after blunt or blast types of TBI by a single systemic dose application (i.v. or i.p.) of a pharmacological “opener” of KCNQ (“M-type”) potassium ion channels in brain.

### 2. Description of the Related Art

Nearly 3 million people in the U.S.A. suffer a traumatic brain injury (TBI) yearly. However, there are no pre- or post-TBI treatment options available to prevent brain damage after a TBI, leading to widespread neurological disease, such as seizures/epilepsy, psychiatric disorders, and suicides. The present invention is the first treatment to prevent these pathological outcomes, and may be applied hours after a TBI is sustained. KCNQ2-5 voltage-gated K⁺ channels underlie the neuronal “M current,” which plays a dominant role in the regulation of neuronal excitability. Prevention of TBI-induced brain damage is predicated on the hyper-excitability of neurons induced by TBIs and the decrease in neuronal excitation upon pharmacological augmentation of M/KCNQ K⁺ currents.

### BRIEF SUMMARY OF THE INVENTION

The present invention is a method for the prevention of brain damage and dysfunction after blunt or blast types of traumatic brain injury (TBI) by a single systemic dose application (i.v. or i.p.) of a pharmacological “opener” of KCNQ (“M-type”) potassium ion channels in brain. Such brain damage and dysfunction can include post-traumatic seizures, a mal-adaptive inflammatory response, microgliosis, astrogliosis, the widespread neuronal death, breakdown of the blood-brain-barrier, post-traumatic epilepsy, cognitive and locomotor dysfunction, mood changes, early Alzheimer's Disease and loss of quality of life.

The present invention does not concern one particular opener molecule, but rather to the approach that is most likely applicable to many of them. Such opener molecules include retigabine (RTG), (2-amino-4-(4-fluorobenzylamino)-1-ethoxycarbonylaminobenzene, an aminoalkyl thiazole derivative (known as Ezogabine in Europe and sold under the trade name, Potiga, formerly FDA approved but now off the market as an anti-convulsant), ICA-069673 ([N-(2-chloro-5-pyrimidinyl)-3,4-difluorobenzamide]) and ICA-27243, and its derivatives, (2-amino-4-(arylamino-phenyl) carbamates (SF0034, RL648_81), and its derivatives, and other such “openers” (agonists) of KCNQ2-5 potassium ion channels that increase their currents in brain neurons.

Seizures are very common after a TBI, making further seizures and development of epilepsy disease more likely. The present invention demonstrates that TBI-induced hyperexcitability and ischemia/hypoxia lead to metabolic stress, cell death and a maladaptive inflammatory response that causes further downstream morbidity. Using a mouse controlled closed-cortical impact blunt TBI model, and the “blast-tube” TBI model, systemic administration of the prototype M-channel “opener,” retigabine (RTG), 30 min after TBI, reduces the post-TBI cascade of events including spontaneous seizures, enhanced susceptibility to further seizures, metabolic stress, harmful inflammatory responses, blood-brain barrier (BBB) breakdown, cell death and development of long-term epilepsy disease.

There are currently no treatments to prevent brain damage after a TBI, leading to widespread neurological disease, such as seizures/epilepsy, psychiatric disorders, and suicides. The present invention is the first treatment that will prevent these pathological outcomes, and may be applied hours after a TBI is sustained.

Currently, there are no medical treatments for either kind (blunt or blast) of TBI whatsoever, and thus, widespread mortality and morbidity numbering in the millions is the result. Thus, the present invention is the first treatment after a TBI event to prevent brain damage and dysfunction for millions of Americans and tens of millions of individuals worldwide. Thus, the present invention represents a completely new and novel approach for treatment of TBI that has been tested by no other lab or entity, worldwide.

### BRIEF DESCRIPTION OF THE SEVERAL VIEWS OF THE DRAWINGS

FIG. 1 is a schematic of the chain of events of an embodiment of the present invention.

FIG. 2 depicts general characteristics of M channels as used in the present invention.

FIG. 3 shows graphs tracking action potential firing in response to various stimuli as used in the present invention.

FIGS. 4A and 4B depict graphs and electroencephalography recordings, respectively, documenting experimental results of the present invention.

FIGS. 5A and 5B show action potential recordings and resulting data, respectively, of the present invention.

FIG. 6 shows a graph documenting experimental results of the present invention.

FIG. 7 depicts a graph documenting experimental results of the present invention.

FIG. 8 depicts a graph documenting experimental results of the present invention.

FIG. 9 shows graphs and in vivo images, respectively, documenting experimental results of the present invention.

### DETAILED DESCRIPTION OF THE INVENTION

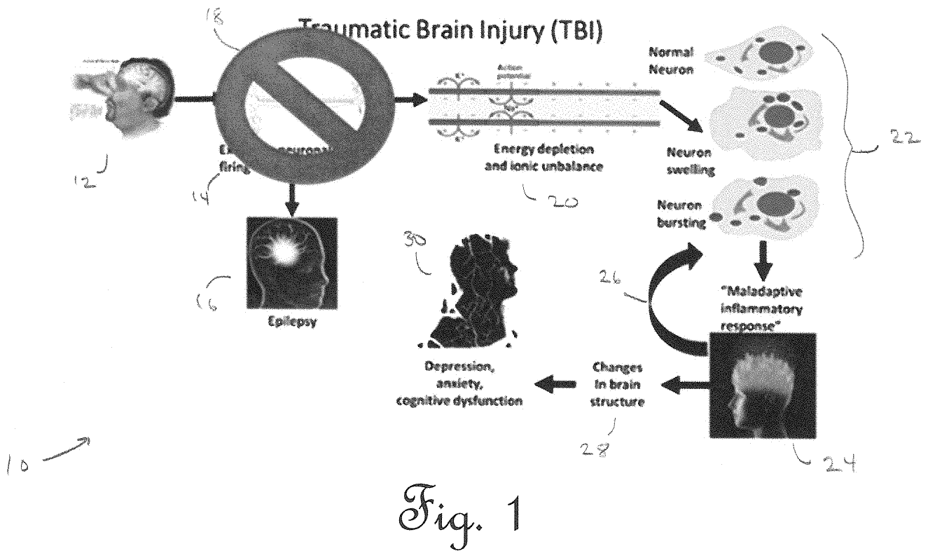

FIG. 1 depicts the chain of events 10 of the present invention. An example of a traumatic brain injury may be as a result from the impact of a fist to the front of someone's head 12. This TBI may result in a concussion and also post concussive syndrome with significant headaches, and short term memory loss. Excessive neuronal firing 14 commences. If continued, then post-traumatic seizures and epilepsy 16 may result. If, however, excessive neuronal firing 14 does not cease, next follows the depletion of energy and ionic unbalance event 20. This may continue until normal neurons begin to swell and ultimately burst, as shown in event 22 in FIG. 1. A maladaptive inflammatory response 24 occurs. From here, either further swelling and bursting of neurons may continue, as shown by return loop 26. Changes in the brain structure and proper functioning 28 become evident, leading to the final event 30 of depression, anxiety and cognitive dysfunction.

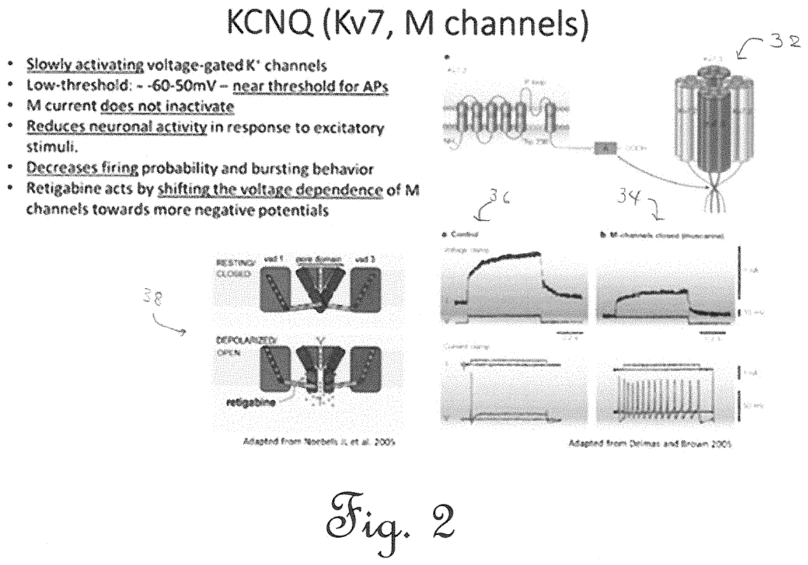

Referring now to FIG. 2, the general characteristics of M channels 32, which are tetramers of four subunits, most often KCNQ2 & KCNQ3. It also shows current waveforms 34 and 36 before and stimulation of muscarinic acetylcholine receptors (top) 34 and the greatly induced action potential (AP) firing 36 when M current is reduced as shown.

Slowly activating voltage-gated K⁺ channels have a low threshold of ˜−60 to −50 mV—near the threshold for action potentials. The M current 32 does not inactivate, but instead reduces neuronal activity in response to excitatory stimuli which decreases firing probability and bursting behavior. Retigabine acts by shifting the voltage dependence of M channels towards more negative potentials, as shown in representation 38 of FIG. 2.

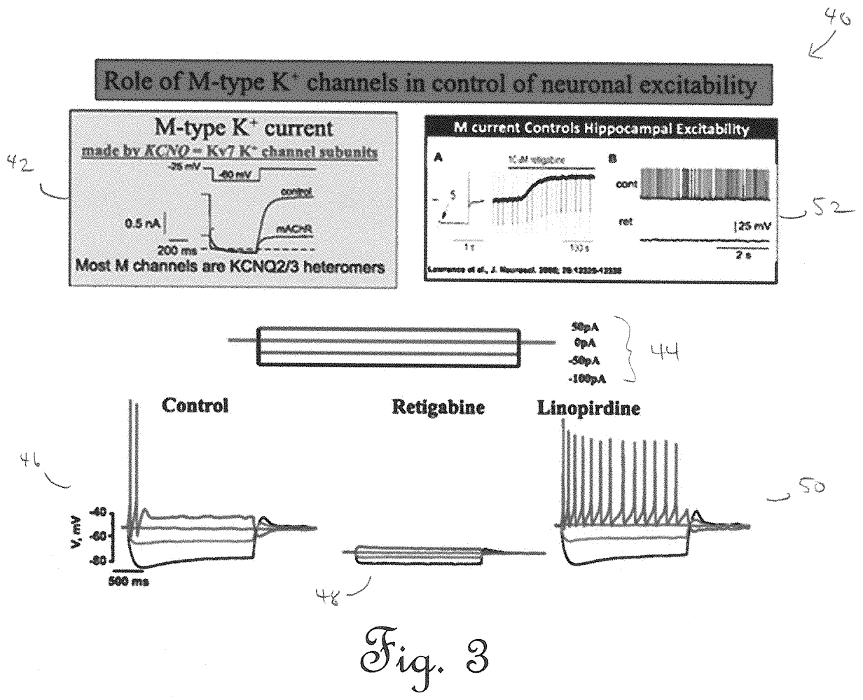

Turning now to FIG. 3, the role 40 of M-type K⁺ channels in control of neuronal excitability is discussed. M-type K⁺ current is provided by KCNQ Kv7 K⁺ channel subunits, as depicted by representation 42. Various currents are applied 44 ranging from 50 pA to −100 pA, and the resulting action potential firing recorded.

Graph 48 shows action potential (AP) fires in response to stimuli in a sympathetic neuron when M current is enhanced by the “opener” drug retigabine. Complete silencing of the neuron occurs, as shown by graph 48. When M channels are completely blocked by the M-channel “blocker,” linopirdine, the result is out-of-control AP firing as when a seizure occurs, or when retigabine is applied to a “brain slice” of the hippocampus in brain, as shown by graphs 52, again converting a rapidly-firing neuron into one totally quiescent. Thus, manipulation of M-channel activity profoundly affects excitability, and a pharmacological “opener” can act as a potent brake on hyperexcitability.

The present invention suggests that acutely reducing neuronal excitability and energy demand via M-current enhancement is a novel model of therapeutic intervention against post-TBI brain damage and dysfunction. This is done by reducing the initial TBI-induced hyperexcitability before seizures occur that only exacerbate cellular energy depletion, thus, “nipping in the bud” the deleterious chain of events that cause a TBI to be so damaging to the brain (see, e.g., FIG. 1).

As discussed further below, the newer M-channel opener, RL-648_81, applied 30 min after a blunt TBI, reduces the increased seizure susceptibility in brain slices, ex vivo. RL-648_81 also prevents the TBI-induced hyper-excitability in cortex when neuronal activity is imaged in vivo through a cranial window in a living mouse brain. Finally, experimentation demonstrates that brain damage occurs after 3-consecutive mild blast TBIs, and that such is prevented by M-channel augmentation, a series of events often experienced by members of the armed services. Administration of an M-channel opener once after each mild TBI will abolish the deleterious effects on the brain.

An additional part of this invention concerns the effects of, and prevention of, repetitive blast TBI on post-trauma seizures and neuronal hyper-excitability in brain. Repetitive blast traumatic brain injury (TBI) affects a large number of soldiers on the battlefield. Mild TBI has been shown to have long-lasting effects if it is a repeated injury. Effects on neuronal excitability are shown after repetitive mild TBI in a mouse model of blast-induced brain injury. Thus, mice were exposed to mild blast trauma of an average peak overpressure of ˜15 psi, repeated across three consecutive days. Whereas a single exposure did not reveal trauma as indicated by the glial fibrillary acidic protein (GFAP) indicator, three repetitive blasts did result in significant increases in brain dysfunction.

In addition, mice had an increased indicator of inflammation (the biomarker Iba-1) increased tau expression, tau phosphorylation, tau being a marker for early Alzheimer's disease, and altered cytokine levels in the spleen. Video electroencephalographic (EEG) monitoring 48 h after the final blast exposure demonstrated seizures in 50% (12/24) of the mice, most of which were non-convulsive seizures. Long-term monitoring revealed that spontaneous seizures developed in at least 46% (6/13) of the mice. Patch clamp brain-slice electrophysiological recording of dentate gyms (DG) hippocampus neurons 48 h post-blast TBI demonstrated a shortened latency to the first spike and hyperpolarization of action potential threshold. Evoked excitatory postsynaptic current amplitudes were also significantly increased.

These findings indicate that mild, repetitive blast exposures cause increases in neuronal excitability and seizures and eventual epilepsy development in some animals. The non-convulsive nature of the seizures suggests that subclinical seizures may occur in individuals experiencing even mild blast events, if repeated, representing a harbinger of developing clinical seizures and epilepsy disease.

In experiments, the present invention modeled mild blast TBI injury in mice to observe effects on neuronal excitability. Mice were exposed to an average of 14.6±0.5 psi (100 kPA±3.4 kPa) maximum peak overpressure, which has been classified as mild blast TBI (0-145 kPa) in rats and mice using a number of physiological parameters. Experiments were first performed to evaluate the effect of the blast TBI model, with a single exposure, or with repeated second and third exposures using 24-hour intervals between TBI.

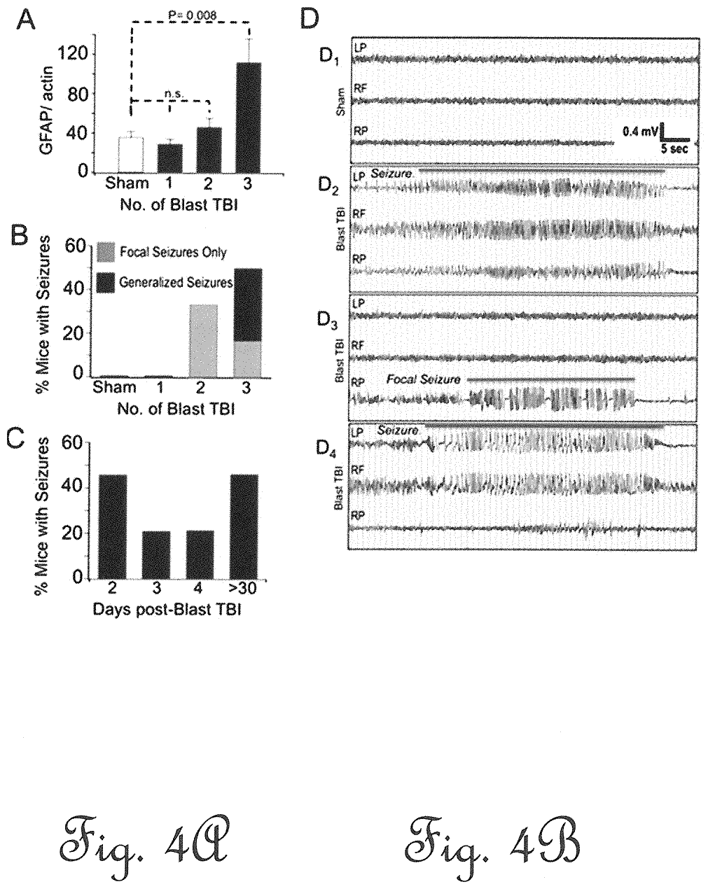

Referring now to FIGS. 4A and 4B, as an indicator of TBI severity, GFAP protein levels were assayed. With reference to FIG. 4AA, GFAP expression increases with repetitive Blast-TBI injuries. Measurements were made from the rostral region (prefrontal cortex) of the brain. GFAP/actin expression is shown for: sham N=5, 35.8±5.9; single TBI (1) N=5, 29.0±5.0; double TBI (2) N=5, 46.2±9.0; and triple TBI (3) N=3, 112.0±24.1, blast TBI. Significance was only observed comparing Sham to 3 Blast TBI, P=0.008.

With reference to FIG. 4AB, the incidence of seizures in mice after sham (N=7) was 0%, single TBI (1) was 0%, double TBI (2) was 33% (2/6 focal seizures), and triple TBI (3) was 50% (3/24 focal, 9/24 generalized seizures). The % of mice with seizures (grey is focal, and black is generalized) is plotted. Data was measured from N=7 sham, 6 single blast, 6 double blast, and 24 triple blast TBI.

With reference to FIG. 4AC, the triple blast TBI mice were measured for incidence of seizures after 2 days (N=24 mice), 3 days (N=19 mice) and 4 days (N=14 mice) following blast TBI. Additionally, spontaneous seizures were detected after 30 days post-TBI (N=6/13 mice displayed spontaneous seizures).

With reference to FIG. 4BD, examples of EEG recordings from Sham (D1) and blast TBI-subjected mice (D2 and D3). Examples of acute post-TBI generalized (D2) seizure or focal (D3) seizure (D4). Example of a spontaneous seizure >30 days post-TBI showing right frontal (RF), right parietal (RP), and medical parietal (MP). Seizure events are indicated with red lines.

The GFAP protein levels are established to be elevated in numerous TBI models, even in mild TBI. A single or double blast injury did not result in significant elevations of GFAP (assayed seven days after last injury) using western blot detection (FIG. 4AA). However, three repetitive blast waves occurring over 3 days did show significant elevations of GFAP expression (FIG. 4AB). These findings show that blast injury in mice produces profound electrophysiological consequences, if repeated (FIG. 4AC). Repetitive mild blast injury causes non-convulsive seizures (50% of mice). Monitoring of mice for a minimum of 10 months after initial blast trauma revealed significant epilepsy development (46% of mice) (FIGS. 4AB-4BD).

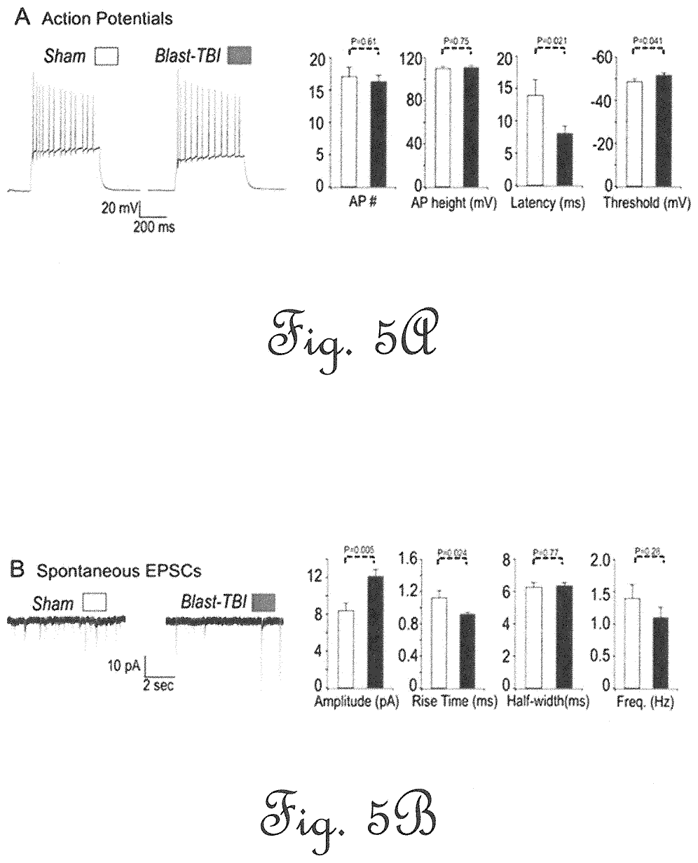

Referring now to FIG. 5A, at the top left, sample action potential (AP) recordings from a sham mouse and one that received 3 repetitive blast injuries (Blast) are shown. APs were measured using whole cell recordings of DGGCs, evoked by a 200 pA current injection. A summary of the data is also shown in FIG. 5A at the top right. Summarized, the data includes: AP number (per 500 ms current injection): 17.1±1.3 sham, 16.3±0.9 TBI; AP height: 110.0±1.6 mV sham, 110.7±1.7 mV TBI; AP latency: 14.0±2.6 ms sham, 8.1±1.1 ms TBI; AP threshold: −48.6±1.2 mV sham, −51.7±0.9 mV TBI.

Referring now to FIG. 5B, blast TBI increases the amplitude and decreases rise time of EPSCs. In the left panel, sample recording of spontaneous excitatory post-synaptic currents in DGGCs are shown. EPSCs were measured at −80 mV holding potential. A summary of the data is shown in the right panel of FIG. 5B. Summarized, the data includes: EPSC amplitude: 8.4±0.8 pA sham, 12.1±0.7 pA TBI; EPSC rise time: 1.1±0.08 ms sham, 0.92±0.02 ms TBI; half-width: 6.3±0.3 ms sham, 6.4±0.2 ms TBI. For the above EPSC data, the frequency was 1.4±0.2 Hz (sham), 1.1±0.1 Hz (TBI).

Immunostaining identified the dentate gyms of the hippocampus as one brain region with elevations of markers for inflammation (Iba1) and astrogliosis (GFAP). Brain slice recordings of dentate gyms neurons 24 hours after blast injury revealed pro-excitatory effects including a reduced threshold and latency for action potential generation in dentate gyms granule cells (DGGCs) (FIG. 5A), and larger post-synaptic excitatory currents (FIG. 5B). Despite the robust EEG electrophysiological consequences, it was discovered that the seizures lack motor abnormalities suggesting that individuals with blast-induced TBI may experience frequent post-trauma seizures that are undetected and untreated. M-channel openers will prevent these harmful effects.

Given that the blast wave was directed head-on to the mice, it was to be determined if blast-TBI trauma was localized to specific regions of the brain. To accomplish this, the brain was dissected into 3 mm sections of the rostral, medial and caudal portions, and conducted western blot detections of GFAP as a marker of trauma. Significant increases in GFAP expression in medial and caudal portions were found. These results are consistent with blast injury causing astroglial activation in broad regions of the brain, which is detrimental to brain health.

It has recently become evident that increased tau expression, associated with Alzheimer's Disease, commonly accompanies both traumatic brain injury, and epilepsy, and tau protein might contribute to seizures. Changes in tau protein were, therefore, investigated, and tau hyperphosphorylation in mice subjected to blast TBI. There was a significant increase in total tau expression found preferentially localized to caudal regions of the brain. Hyperphosphorylation of tau was assayed by normalizing phospho-tau proteins to total tau protein. It was found that blast trauma increased phospho-tau in the middle (Ser202) and rostral regions (Ser396/Ser404). Given that total tau protein was elevated in the caudal regions of the brain, it was also investigated if phospho-tau could be elevated due to increased levels of tau protein in this region. phospho-tau was, therefore, normalized to GAPDH, rather than total tau. It was indeed found that Ser202 was significantly elevated in the caudal region when normalized to GAPDH.

These findings are the first experimental evidence indicating that mild, repetitive blast exposures cause increases in neuronal excitability and seizures, and eventual epilepsy development in some animals. The results also suggest that blast-injury epilepsy can be modeled in mice, allowing support of this discovery via changes in excitability both in the whole animal and at the level of single neurons. The predominantly non-convulsive nature of the seizures also suggests that subclinical seizures may occur in individuals experiencing even mild blast events, if repeated. The present invention may prevent deleterious effects on the brain of such repetitive events.

In another embodiment of the present invention, the present invention pertains to the long-term effects of TBI, in particular the development of epilepsy disease, which is wholly distinct then treating seizures with anti-convulsants. In the blast TBI model, 67% of mice displayed spontaneous seizures, as observed by electroencephalogram (EEG) and video recording from the period of 1 month to 12 months after injury. The occurrence of these seizures confirms the occurrence of post-traumatic epilepsy (PTE) after Blast-TBI. RTG treatment was able to significantly reduce PTE occurrence after Blast-TBI. Only 25% of the RTG treated mice developed PTE. Eight sham animals were also monitored and no seizures were observed. There is currently no FDA approved treatment for PTE. Hence, treatment with M-channel openers represent a paradigm change in PTE prevention.

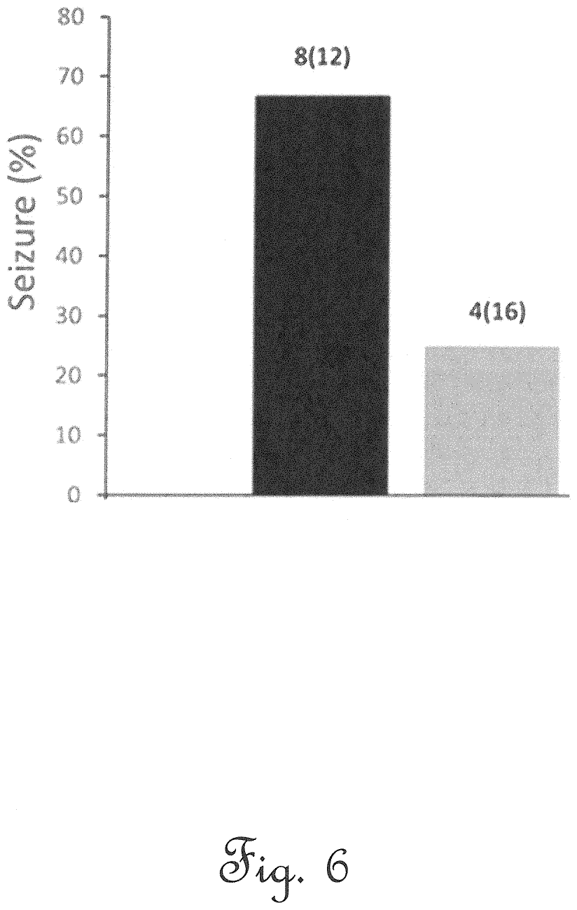

Referring now to FIG. 6, the results of this analysis is illustrated, along with that of a stimulator of astrocyte metabolism, as a control. Clearly, RTG was responsible for the protective effect, consistent with the present invention.

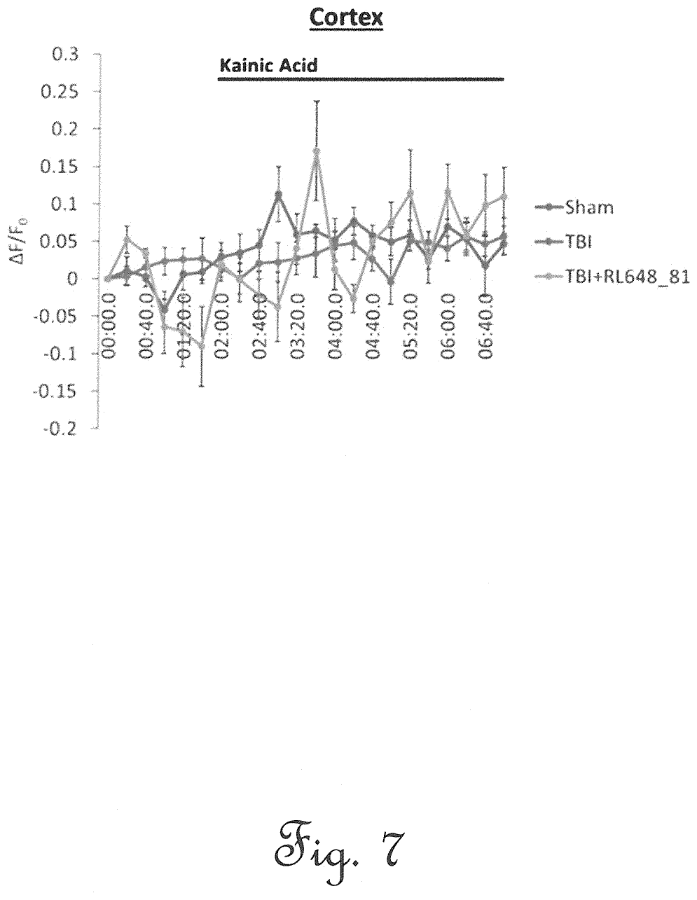

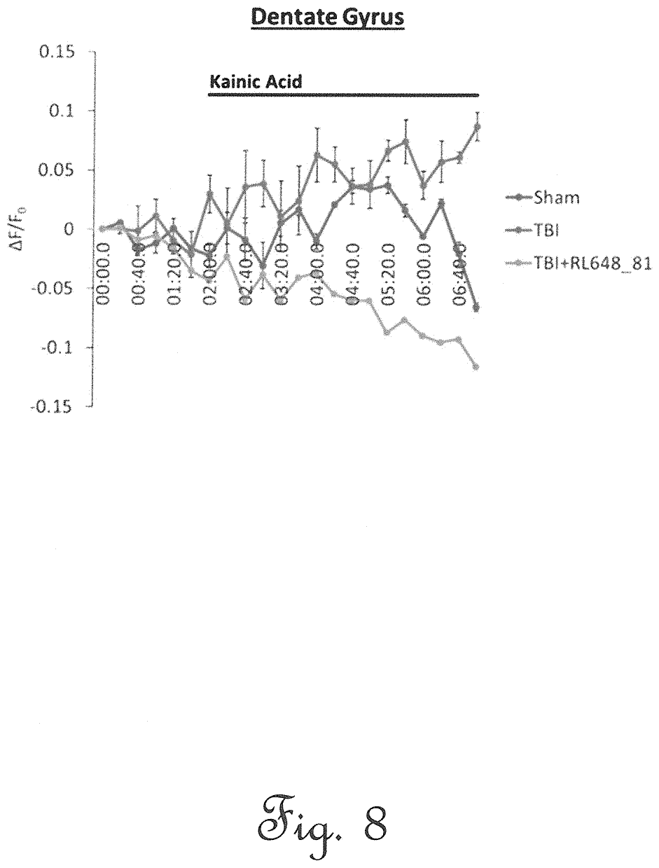

In another embodiment, the present invention concerns the ex vivo imaging of intracellular calcium levels on neurons in the brain slice. Now with reference to FIGS. 7 and 8, transgenic mice were used to image intracellular [Ca²⁺] inside brain neurons using ex vivo brain-slice imaging experiments. The mice used were made to express the fluorescent calcium reporter, GCaMP6f, under the control of the Thy1-Cre promoter, which drives expression in critical subsets of brain neurons. Hence, intracellular [Ca²⁺] could be imaged in neurons of the cortex and hippocampus in these mice. Changes in intracellular [Ca²⁺] are a reporter of changes in neuronal firing and excitability.

In the first pilot experiments, the chemoconvulsant, Kainic Acid (KA), was used to stimulate neurons from brain slices of the cortex and in the dentate gyms (DG) of the hippocampus (the part of the brain where most new memories are thought to form) from sham, TBI+vehicle and TBI+RL-648_81 cohorts. RL-648_81 is a new generation drug that facilitates M-current solely produced by heteromeric tetramers of KCNQ2&3 subunits, thus sparing side-effects on smooth muscle, such as that in bladder, which cause urinary incontinence.

Based on the EEG results presented previously, it was discovered that M-current facilitation by RL-648_81 treatment impairs TBI-induced increased hyperexcitability in response to KA stimulation. As can be observed below, the experiments show DG to have a stronger TBI-induced increase in the response to KA stimulation, which was completely abolished by RL-648_81 treatment 30 min after the TBI. Thus, the present invention concerns not only one molecule, but rather the general approach of preventing TBI-induced brain damage, which has never before been postulated or demonstrated.

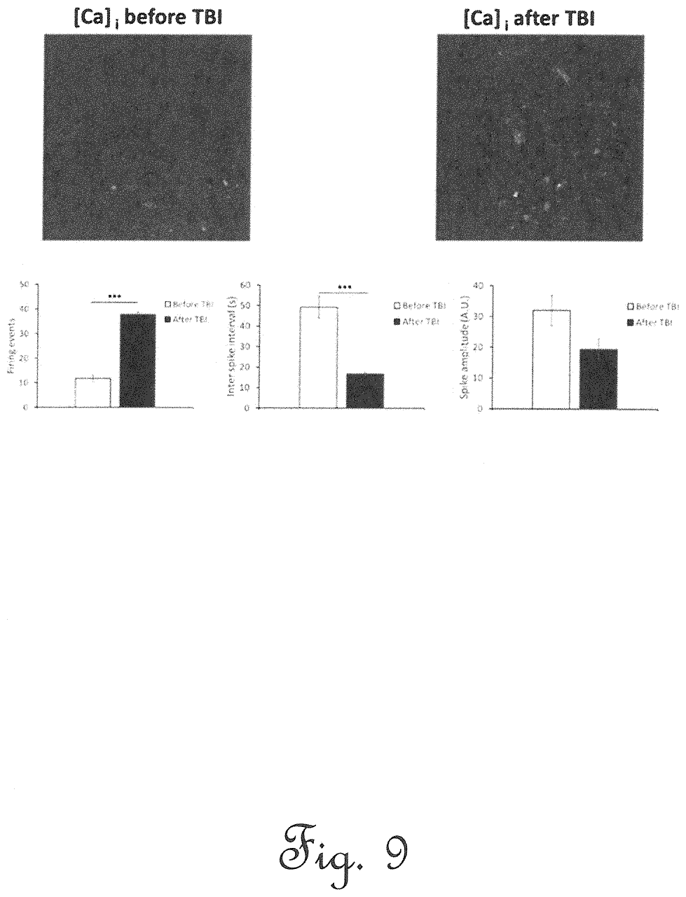

In another embodiment, the present invention concerns in vivo imaging of intracellular [Ca²⁺] on living brain in vivo, as a reporter of activity/hyperactivity. The genetically-encoded Ca²⁺ reporter, GCaMP6s, was also used expressed in transgenic mice mated with Thy1-Cre mice, to do in vivo recordings of living brain in vivo, as a reporter of activity/hyperactivity in cortex induced by TBI, the first event in the hypothesis of TBI-induced brain damage. For these experiments two-photon confocal live imaging of mouse cortical neurons through a cortical window was used. Live video records of the activity of cortical neurons in mice before and after TBI were made. As typified in FIG. 9, a significant increase in neuronal firing and a decrease in inter spike interval was observed after a blunt TBI.

Brain damage, deficits and dysfunction resulting from commonly occurring TBIs can be prevented by acute administration of a KCNQ/Kv7/M-type potassium channel opener shortly after the occurrence of a TBI. The TBI can be of the blunt type or the blast/shockwave type, as commonly experienced in falls, impact of the head with a hard object, vehicular accidents, explosions on the battlefield in the vicinity of military service personnel, or shock waves from the use of large weapons near the victim by either a fellow service member, or the enemy. Even very mild TBIs, although not perceptible to the victim, when repeated multiple times, cause brain damage, which can also be prevented by M-channel pharmacological openers when administered after each TBI, or after a succession of TBIs.

The present invention has application in the treatment of brain injuries caused as a result of engaging in contact sports/athletic events, in which head impacts are common, including American football, soccer, ice hockey, boxing, bicycle falls and the like.

The various embodiments described herein may be used singularly or in conjunction with other similar methodologies. The present disclosure includes preferred or illustrative embodiments in which a method for the prevention of brain damage and dysfunction after blunt or blast types of TBI by a single systemic dose application (i.v. or i.p.) of a pharmacological “opener” of KCNQ (“M-type”) potassium ion channels in brain is described. The present invention is intended to cover the entire spectrum of M-channel “openers,” including those listed herein, and any new compounds or derivatives of existing compounds that may be developed on this same target. Alternative embodiments of such a method can be used in carrying out the invention as claimed and such alternative embodiments are limited only by the claims themselves. Other aspects and advantages of the present invention may be obtained from a study of this disclosure and the drawings, along with the appended claims.

I claim:

1. The method of preventing brain damage and dysfunction after traumatic brain injury, said method comprising the steps of:
administering a dose of a pharmacological opener to an organism;
monitoring said organism for a predetermined time;
observing neuronal activity; and
recording said neuronal activity.

2. The method of claim 1 wherein in said administering step, said pharmacological opener is a KCNQ/Kv7/M-type potassium channel opener.

3. The method of claim 2 wherein said administering step is performed shortly after the occurrence of a traumatic brain injury.

4. The method of claim 3 wherein the neuronal excitability and cellular energy demand are reduced via M-current enhancement.

5. The method of claim 4 wherein said dose in said administering step is limited to a single dose.

6. The method of claim 5 wherein said traumatic brain injury is of blunt traumatic brain injury.

7. The method of claim 5 wherein said traumatic brain injury is of blast traumatic brain injury.

8. The method of claim 6 wherein in said administering step, said pharmacological opener is retigabine and its derivatives.

9. The method of claim 7 wherein in said administering step, said pharmacological opener is retigabine and its derivatives.

10. The method of claim 6 wherein in said administering step, said pharmacological opener is RL648_81 and its derivatives.

11. The method of claim 7 wherein in said administering step, said pharmacological opener is RL648_81 and its derivatives.
